(19) United States
(12) Patent Application Publication
(10) Pub. No.: US 2009/0216214 A1
(43) Pub. Date: Aug. 27, 2009

(54) Therapeutic agent treat to target system

(76) Inventors: Burton H. Sage, Carlsbad, CA (US); Bruce Michael Burgess, Coto De Caza, CA (US); Michael Albert Klimowicz, Escondido, CA (US)

Correspondence Address: BURTON H. SAGE, JR., 2453 IMPALA DR., CARLSBAD, CA, 92010, US

(21) Appl. No.: 12/001,252

(22) Filed: Dec. 12, 2007

Related U.S. Application Data
(60) Provisional application No. 60/931,758, filed on May 29, 2007.

(51) Int. Cl. A61M 5/31; A61M 37/00

(52) U.S. Cl. 604/504; 604/290

(57) ABSTRACT

A method of calculating doses of insulin for a person with diabetes and administering the calculated doses to the person with diabetes is presented. Blood glucose values are provided to an insulin dosing calculator which implements a Treat to Target protocol to calculate appropriate doses of insulin. An insulin dispenser is also provided to fill an insulin administration device with the calculated dose.

This application claims subject matter disclosed in copending application No. 60/931,758 dated May 29, 2007 entitled Passive Drug Delivery system, the contents of which are incorporated herein in their entirely by reference.

### BACKGROUND

This application relates to methods of administrating medicinal therapies and in particular to a method of administering insulin according to a protocol to rapidly and safely reach a targeted blood glucose level.

Insulin is a particularly difficult medicine to administer. Not only does the proper dose of insulin vary from person to person, the proper dose also varies from day to day and dosing time to dosing time depending on the food to be ingested and the amount of physical exertion the person has expended during the course of the day. Hence the amount of insulin to be administered at any given time must be determined using a number of different factors. The correct amount varies from a low of a few units in any one administration, which may be give up to 4 times per day to over 150 units per day. This situation is further exacerbated by the fact that insulin is not bioavailable when taken by mouth. Since its discovery in 1922, only two methods of administration of insulin have proved practical. The first method is by syringe and vial, where the person withdraws the desired amount of insulin from a vial into a syringe and injects the desired dose under the skin. The second method, available only for the last ten to fifteen years, is the insulin pen where the insulin is contained in the pen and the user injects a selected dose under the skin by selecting the desired dose using a dial on the pen. In both of these methods, the amount of insulin is selectable by the unit, that is, over the range of 10 units to 150 units, the dose is selected to the nearest unit.

In selecting a dose of insulin, either the patient or the physician in charge of the management of the patient's diabetes must make a determination of the proper size of a dose. With rare exception, on a day to day basis, the patient is the person who makes this decision. While the physician may be best qualified, he neither has the time himself nor other resource to make this decision for the patient on a day to day basis. Nor is the physician compensated for making this dosing decision other than a periodic fee for an office visit. The patient may or may not be motivated to make a dosing decision, and he is usually the least skilled or qualified. Methods to help the patient decide on a proper dose include counting carbohydrates and making calculations based on measurements of blood glucose. For the large part, though, these calculations are difficult to make, error prone, and even when done properly do not provide sufficiently accurate control of blood glucose levels. Proper dosing of insulin for a person with diabetes to adequately control their blood glucose is far from routine and compliance to any regimen is poor.

Over the past several years an approach generally called “Treat to Target” has been studied for use primarily in persons with Type II or non-insulin dependent diabetes. An example of such a study is found in Alvarsson, M et. al. “Beneficial Effects of Insulin versus sulphonylurea on insulin secretion and metabolic control in recently diagnosed Type II Diabetic Patients”, Diabetes Care 26, 2231-2237, 2003.

In one common version of this method a person measures their fasting blood glucose in the morning and injects a dose of long acting insulin, typically Lantus (Hoechst-Aventis) or Levemir (Novo) based on that glucose measurement. The doses of insulin are increased periodically until the fasting blood glucose reaches a target, typically 100 mgm % (5.5 mMolar). Once that target is reached, the dose is then held constant until there is some further change in the fasting blood glucose level. Large, multi-center clinical studies have shown that this method is very effective in reducing fasting blood glucose, and does so more quickly and safely compared to other therapeutic regimens (for example, the combination of a sulphonylurea and metformin). Further, the “Treat to Target” method, when used in newly diagnosed persons with diabetes and who are treatment naïve, restores a large fraction of the person's normal insulin secretion capability (see: The effect of intensive insulin therapy on pancreatic beta cell function in newly diagnosed Type 2 diabetic patients—Poster, Annual American Diabetes Association Meeting, Chicago, 2007). In studies of this method, though, the interaction between the person with diabetes and a healthcare professional is intense with an interaction at least once a week and often twice a week to encourage compliance and to monitor use of the protocol. To treat the tens of millions of person with Type II diabetes in the world today, there is insufficient manpower to implement a Treat to Target method. Hence there is a clear need for a method of implementing a Treat to Target protocol that does not require intensive interaction with a healthcare professional and does not require a high level of skill from the person with diabetes.

### SUMMARY OF THE INVENTION

In one aspect of the invention a method of implementing a Treat to Target protocol for managing blood glucose levels that requires minimal time of the healthcare professional and a minimum of skill from the person with diabetes or the person caring for the person with diabetes is provided. The only requirement of the healthcare professional is the diagnosis of diabetes and the providing of a prescription for insulin. The only requirement of the person with diabetes or the person caring for the person with diabetes is the ability to obtain a measurement of glycemic control when requested and to enter that measurement value, either by using a keyboard, or by saying the glucose value (to be interpreted by a voice recognition and verification system), or by using a cable or wireless transmission from the glucose meter. The measurement of glycemic control may be a fasting blood glucose value, or a fasting measurement of glucose concentration in interstitial fluid as is now performed by several continuous glucose monitoring systems such as the Navigator™ manufactured by Abbott Laboratories. The measurement of glycemic control may also be a fructosamine value, a hemoglobin A1C value, or any value shown to adequately represent the level of glycemic control in the person with diabetes who will receive the insulin dose calculated by this method. In this “Treat to Target” method the measurement of glycemic control is provided to a protocol manager for calculation of an appropriate insulin dose. This protocol manager may be one or more persons or may be completely under the control of software on a computer. The protocol manager uses a Treat to Target protocol and the measurements of glycemic control, which include a recent measurement taken since the last calculated dose of insulin and may include measurements taken on earlier occasions, to calculate a dose of insulin for the person to administer. This insulin dose may be administered one or more times per day depending on the details of the “Treat to target” protocol.

In another aspect of the invention an insulin dispensing unit is provided to fill an insulin administration device with the calculated dose of insulin. The insulin administration device may be a syringe or an insulin pen as is known in the art, or an insulin pump such as the Medtronic Paradigm operating in a “basal only” mode, or an insulin administration patch as described in copending application 60/931,758 dated May 29, 2007 entitled Passive Drug Delivery system, the contents of which are incorporated herein in their entirely by reference. In one aspect of the invention the filling device comprises a container of insulin and a container of diluent. In other aspects of the invention the filling device may be an insulin cartridge with a fixed insulin concentration. Depending on the calculated dose of insulin, varying amounts from each of these containers will be delivered to the insulin administration device such that the volume of fluid put into the insulin administration device has the desired number of units of insulin.

In another aspect of the invention this insulin dispensing device further comprises a communication link from the user to the protocol manager. This communication link may be a land line telephone or a cell phone or an internet connection or any other link capable of accurately communicating the measurement of glycemic control to the protocol manager and to communicate the calculated dose of insulin back from the protocol manager to the insulin dispensing device such that the insulin dispensing device can fill the insulin administration device with the calculated dose of insulin. Alternatively, the protocol manager may comprise computer software that is resident in the insulin dispensing device.

In a further aspect of the invention the insulin dispensing device keeps a detailed record of the amount of insulin dispensed which may or may not be communicated to the protocol manager. By keeping the detailed record of dispensed insulin, the insulin dispensing device is capable of calculating when there is insufficient insulin remaining in the insulin container to provide the calculated dose. In this event, the insulin dispensing device makes the user aware of this lack. The insulin dispensing device may fill the insulin administration device with a partial dose and, when the insulin dispensing device has been resupplied with a new container of insulin, finish filling the insulin administration device, or the insulin dispensing device may simply wait to fill the insulin administration device after the insulin dispensing device has been resupplied with a fresh container of concentrated insulin. Similarly the dispensing device can keep track of the use of the diluent to insure that sufficient diluent is available to fill an administration device and to determine when fresh diluent must be provided. To reduce the chances of infection, each insulin and diluent container system is closed so that the insulin administration device is aseptically filled. Since the protocol manager has a complete record of the doses of insulin that the user has administered, it may also automatically order additional insulin and diluent for the user such that these supplies arrive as they are needed by the user.

### SHORT DESCRIPTION OF THE FIGURES

FIG. 1 shows the organization of the components of the invention

FIG. 2 shows a view of an insulin dispensing device with an insulin delivery patch in position to be filled by the insulin dispensing device.

FIG. 3 shows a view of an insulin delivery patch.

FIG. 4 is a flow chart showing an embodiment of the method when the insulin delivery device is a patch filled by the insulin dispensing device.

FIG. 5 is a flow chart showing the embodiment of the method when the insulin delivery device is a syringe to be filled by the insulin dispensing device.

FIG. 6 is a flow chart showing the embodiment of the method when the insulin delivery device is a syringe that is filled by the person with diabetes or a caregiver for the person with diabetes.

FIG. 7 shows an example of a “Treat to Target” protocol

### DETAILED DESCRIPTION

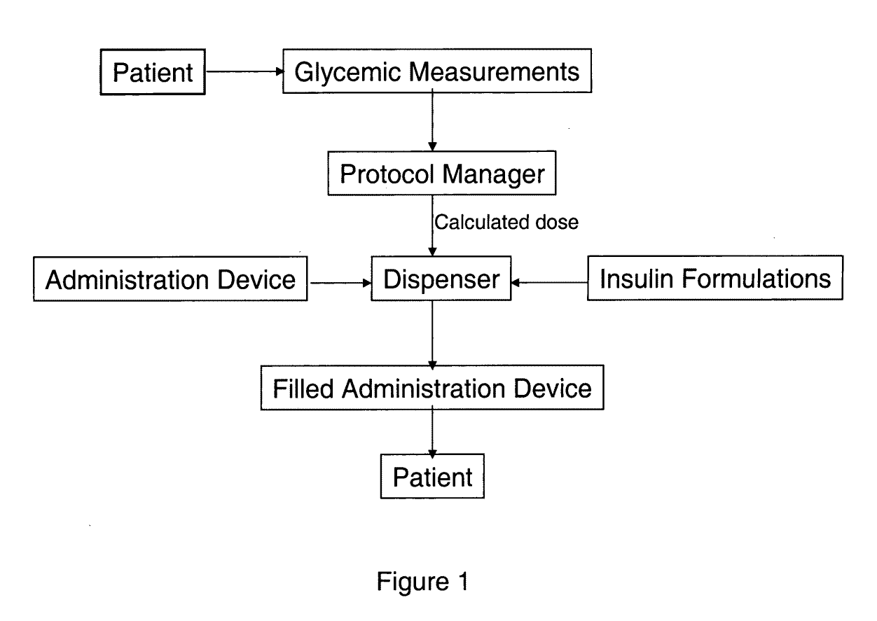

FIG. 1 is a system block diagram of the system of the invention. Use of the system begins with a patient capable of obtaining measurements relating to his glycemic state. Such measurements may be levels of glucose in the blood, or levels of glucose in interstitial fluid as are now possible using continuous glucose monitors available from Dexcom (San Diego) or Medtronic (Minneapolis), or levels of glycated hemoglobin, or may be levels of fructosamine, or levels of any other body analyte which can be related to the patient's glycemic state. In one embodiment of the invention, though, blood glucose is the analyte relating to the glycemic state of the patient.

In this embodiment the glycemic measurements are sent to an entity herein called the protocol manager. The protocol manager may be a person or group of persons, or may be a device such as a computer. This protocol manager is capable of receiving the glycemic measurements and, in one embodiment of the invention, using the glycemic measurements to calculate an appropriate dose of insulin for administration to the patient. The calculated dose may be a basal dose to be administered at a constant rate over a set period of time, for example over the course of an hour, or day, or week. Alternatively, the calculated dose to be administered may be a dose to be given over a very short period of time, for example, a bolus dose of insulin. Or, the dose may be a combination of a basal dose and a bolus dose such that the amount and time of administered insulin has been determined. In this latter case the calculated dose is clearly a dosing regimen, that is, amounts of insulin administered as a function of time.

The calculated dosing information is then sent to a dispensing device. The patient places an administration device such as a patch as described later, or an insulin syringe or an insulin pen. The dispenser then fills the administration device with the appropriate dose as calculated by the protocol manager. As shown in FIG. 1, the dispenser also includes insulin formulations such that the dispenser may fill the administration device with the appropriate dose of insulin. Such insulin formulations include insulin at one or more fixed concentrations or insulin of a fixed concentration and a diluent that includes no insulin. Once the administration device has been filled, the patient removes the filled administration device and uses the administration device to administer the insulin as appropriate. In one embodiment, as shown in detail below, the insulin administration device is a patch which administers insulin at a fixed rate over a fixed period of time. In other embodiments, the administration device is an insulin syringe which has been filled with a calculated dose of long acting insulin such as Lantus (Aventis) or Levemir (novo). Alternatively, the administration device is an insulin pen which has been prefilled with an insulin cartridge. In this case, the dispenser adjusts the dosing increment of the pen to the dose calculated by the protocol manager for the patient. The patient then merely injects the calculated dose.

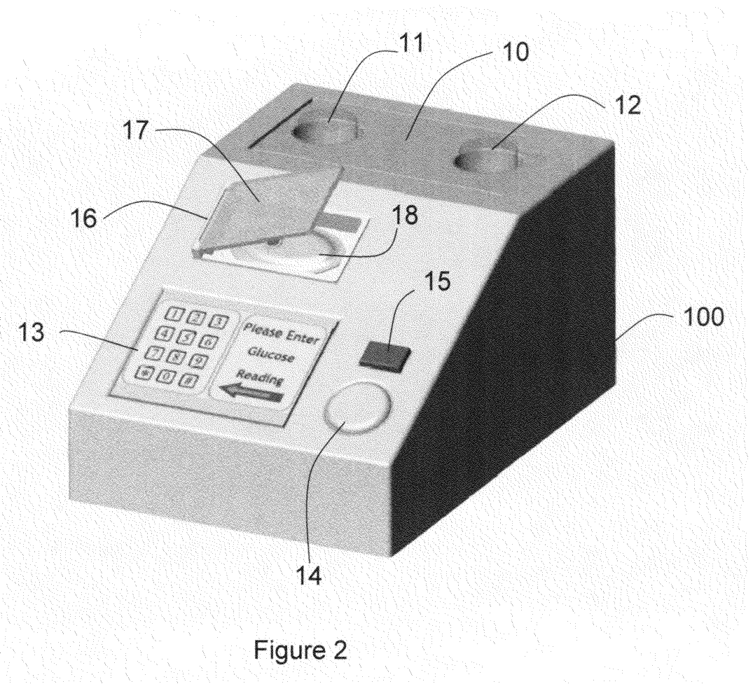

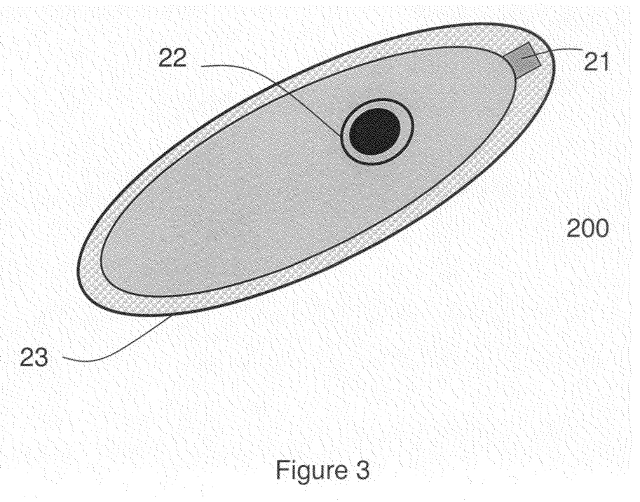

FIG. 2 shows one embodiment of an insulin dispensing device 100. Medication supply cassette 10 is shown inserted into the top of insulin dispensing device 100. Medication supply cassette 10 is further comprised of a vial 11 of concentrated insulin and a vial 12 of diluent for mixing with the insulin from the insulin vial internal to insulin dispensing device 100. Insulin dispensing device 100 further comprises a compartment 16 for placing an insulin administration device which, in this case, is insulin patch 18 shown in more detail in FIG. 3. Compartment 16 further comprises a hinged door 17 to close compartment 16 when insulin patch 18 is being filled by insulin dispensing device 100. In another embodiment not shown the insulin administration device could be an insulin syringe which could alternatively be placed in a similar compartment to be filled by the insulin dispensing device.

Insulin dispensing device 100 further comprises a display and keyboard 13 for displaying instructions and entering information. As shown, insulin dispensing device 100 is requesting a glycemic control value which in this case is a glucose reading. Typically this glucose reading will be obtained using a glucose meter and strip as is well known in the art. This glucose reading is typically a fasting glucose reading obtained first thing in the morning but could be a glucose reading obtained at any other time as required by the details of the Treat to Target protocol as implemented by the protocol manager. Alternatively the glycemic control value could be a fructosamine value or a hemoglobin A1C reading or any other value required by the Treat to Target protocol.

Insulin dispensing device 100 further comprises button 14 which may be used to initiate operation of insulin dispensing device 100, and button 15 which may be used to select menu options to be displayed on display 13.

FIG. 3 shows an artists conception of insulin patch 200 which would be used as the insulin administration device. A more detailed description of one embodiment of insulin patch 200 can be found in application 60931,758 filed on May 29, 2007, the contents of which are incorporated herein in their entirely by reference. Insulin patch 200 has fill port 21 which is used by the insulin dispensing device to fill patch 200 with the calculated insulin dose as determined by the protocol manager. Insulin patch 200 further comprises skin adhesive and release liner 23. After the patch has been filled by the insulin dispensing device, the device is removed from the insulin dispensing device, the release liner 23 is peeled from the adhesive and the device is place on the skin.

Insulin patch 200 further comprises activation button 22. After the adhesive release liner has been removed, the device is place on the skin and activation button 22 is pressed. Pressing the button causes a thin needle (not shown) to be deployed into the skin and causes the insulin dose to start flowing into the skin.

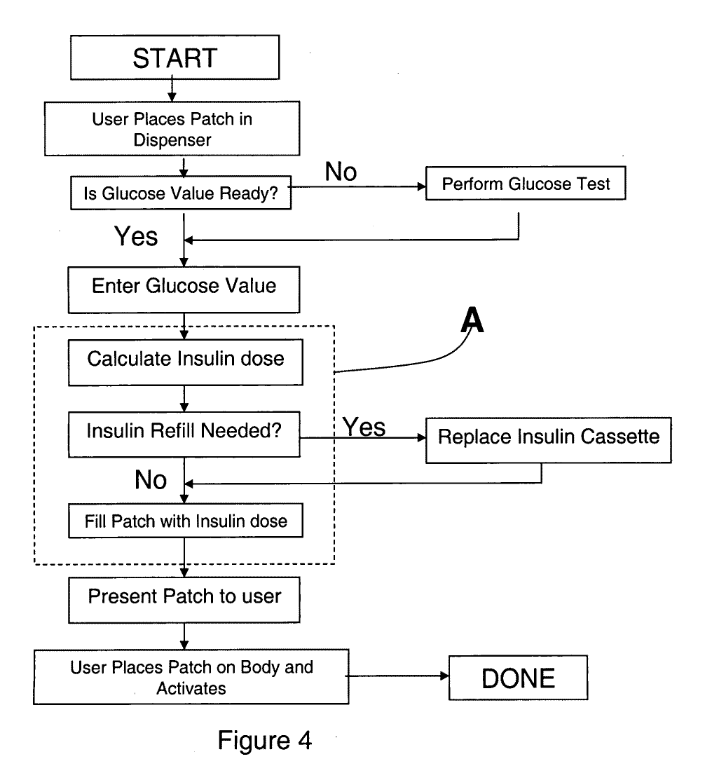

In one embodiment of the invention the user follows the flow chart shown in FIG. 4. In this embodiment the first step is to place an insulin administration device into the insulin dispensing device as shown in FIG. 2. In this embodiment the insulin administration device is a body worn patch into which the calculated dose of insulin will be placed. In other embodiments the insulin administration device could be a syringe or an insulin pen as is well known in the art. When the patch is placed in the dispenser, the user presses the start button and the dispenser requests the user enter a measure of glycemic control, in this case a glucose value. If the user has not yet obtained the glucose value, the user proceeds to obtain the value and enters the value using the dispenser. If the measure of glycemic control is a blood glucose measurement, typically the user will obtain a blood glucose measurement by lancing a finger or forearm to obtain a small drop of blood, putting the drop of blood on a glucose strip, inserting the strip into a glucose meter, and observing the glucose meter display which will show the blood glucose value. The user will then enter the glucose value (the “enter glucose value” box just above the dotted line box in FIG. 4). The mode of entry of the glycemic control measurement, in this case a blood glucose measurement, could be a keypad as shown in FIG. 2 or could be a computerized voice recognition system or any other mode of accurately providing the glucose value in a form accurately recognized by the protocol manager. The blood glucose value is then sent to the protocol manager for processing to calculate an insulin dose (top box in dotted line box A in FIG. 4). In FIG. 4 dotted line box A represents those steps performed by the protocol manager and the insulin dispensing device which is under the control of the protocol manager.

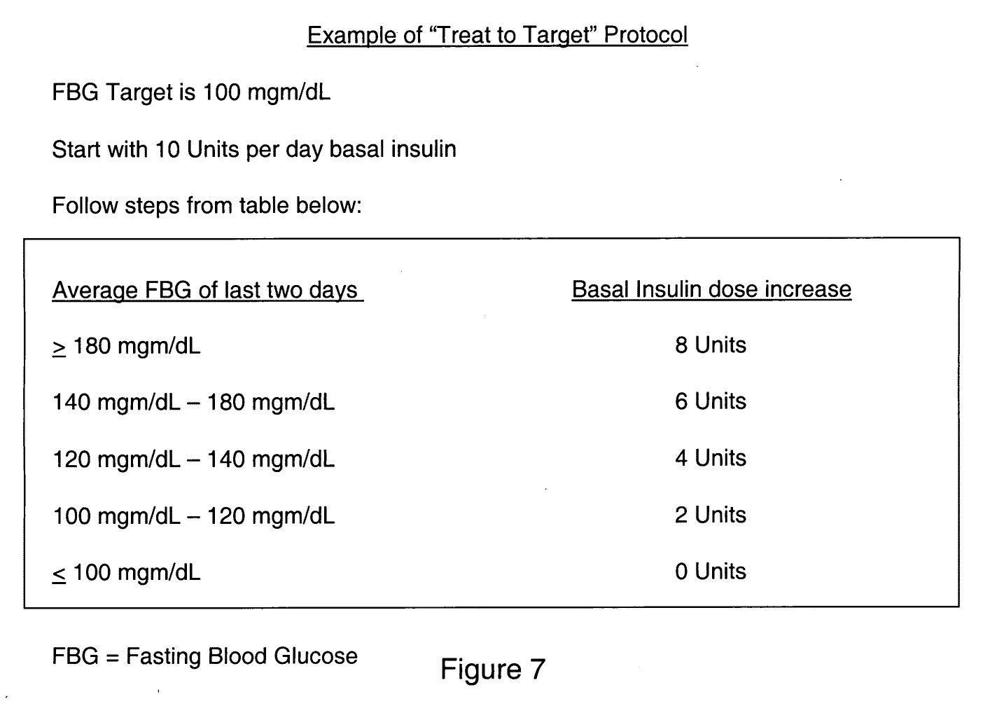

In FIG. 4 the steps included in the dotted line box, denoted by the large capital A, are those steps under the control of the protocol manager. Using the protocol, an example of which is shown in FIG. 7, the protocol manager calculates an insulin dose. Typically the protocol manager would be comprised of a computer system and associated software programmed to accept the glucose value from the dispenser, calculate an insulin dose using the glucose value, and communicate this calculated insulin dose back to the dispenser. Based on the size of the calculated dose and the quantity of insulin remaining in the insulin dispensing device, a determination is made if a new medication cartridge (10 in FIG. 2) is needed. If a new medication cartridge is needed, the insulin refill door on the insulin dispensing device will open and present the spent medication cartridge to the user much like a VHS video cassette is presented to the user after a video tape has been ejected by the VHS video player. The user will dispose of the spent medication cartridge and put a fresh cartridge in the door. The insulin dispensing device will then accept the cartridge, position it for proper filling of insulin administration devices, which in this case is an insulin patch, and complete the filling of the patch.

Since the protocol manager has a record of the doses administered by the user and the amount of medication contained in the medication cartridge when it was first used, the protocol manager can estimate when the user is likely to need a new medication cartridge. In this case the protocol manager can notify an appropriate organization, such as the shipping portion of the company providing the treat to target system, that it is time to resupply the user. The shipping portion of the organization can then fill the resupply order and ship fresh medication cartridges to the user as necessary.

After the patch has been filled, the patch is presented back to the user (Box just below dotted line box A in FIG. 4). The user then removes the patch from the insulin dispensing device, removes the adhesive release liner, places the patch on the body so that the adhesive is in contact with the skin, and presses the activation button on the patch to begin the administration of the insulin. At this point the process is complete. The insulin patch will deliver the insulin at a constant rate over the designed period of time. Since the administration rate is constant, the serum level of insulin will very quickly reach a steady state.

The process shown in FIG. 4 is an embodiment where the insulin delivery device is replaced once a day. Other embodiments of the insulin delivery device and other Treat to Target protocols are possible which comprise insulin dosing episodes longer, for example three days or seven days, or shorter than 24 hours.

In the above-case the insulin administration patch was capable of delivering insulin at a constant rate over the life of the patch. In this case the insulin which would be placed in the patch would be a relatively rapid acting insulin such as regular insulin, or even more rapidly acting insulin such as Novolog (Novo) or Humalog (Lilly) or Apidra (Aventis).

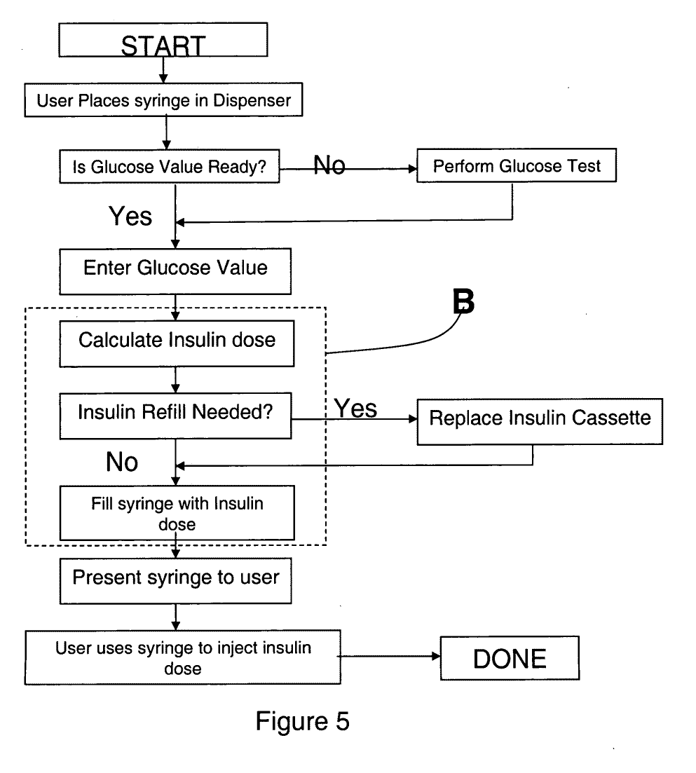

FIG. 5 shows a flow diagram of an embodiment of the Treat to Target system of the invention where the insulin delivery device is a syringe. Since the syringe will provide insulin to the user at only one time, that time being when the syringe needle has actually penetrated the skin of the user, the insulin should be a long acting insulin. Examples of appropriate insulins are Lantus (Aventis) and Levemir (Novo). As in the embodiment described in relation to FIG. 4, the user begins by placing the insulin delivery device, the syringe, in the insulin dispenser (not shown). As before, the user enters a glucose value, whether the user had one before he placed the syringe in the insulin dispenser or the user obtained the glucose value after the placed the syringe in the dispenser. The dispenser then sends the glucose value to the protocol manager where an appropriate insulin dose is calculated (Top box inside dotted line box B in FIG. 5). The protocol manager supplies the calculated dose to the insulin dispenser so that the syringe may be filled with the calculated dose. If the insulin cartridge needs to be replaced, the dispenser will indicate that a new cartridge is needed and the user will replace the cartridge. Note that at this step in the method in both the embodiment shown in FIG. 4 or FIG. 5, the dispenser has maintained a record of the dispensed insulin and hence has calculated the amount of insulin remaining in the dispenser. This information has also been transmitted to the protocol manager so that fresh cartridges of insulin may be sent to the user, usually for storage in a refrigerator, so that a fresh cartridge of insulin is available when the dispenser indicates that one is needed.

The insulin dispensing device then fills the syringe with the calculated dose of appropriate insulin (bottom box in dotted line box B in FIG. 5) and presents it to the user. The user would then inject that dose into the skin.

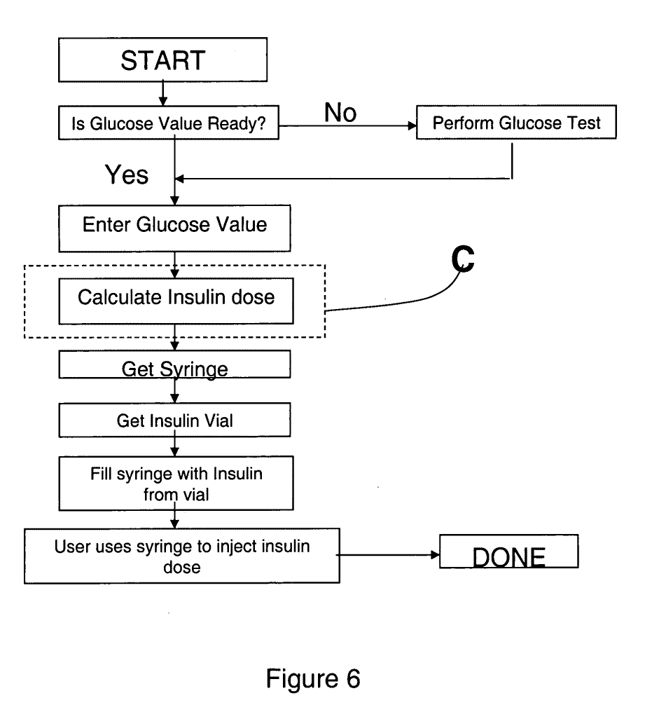

A third embodiment of the invention is shown in FIG. 6. As in the other embodiments, the user obtains a glucose test result and enters that result using the keyboard as shown in FIG. 2. The protocol manager then calculates an appropriate insulin dose. In this case, though, the system does not fill an insulin administration device as was done in the previous embodiment. Instead the user obtains a syringe and a vial of appropriate insulin, fills the syringe with the dose of insulin calculated by the protocol manager, and injects the contents of the syringe into his body. Alternately, if the user has an insulin filled pen, the user would set the pen to deliver the dose calculated by the protocol manager. Once the dose has been set for the pen, the user then administers that dose. This embodiment is especially appropriate for long acting insulins such as Lantus (Aventis) or Levemir (Novo).

I claim:

1. A method of treating a person with diabetes comprising the steps of
a) obtaining a measurement of glycemic control from the person with diabetes,
b) communicating the measurement of glycemic control to a protocol manager,
c) using the protocol manager to calculate a dose of insulin based on the obtained glycemic control measurement and previously communicated glycemic control measurements if earlier obtained,
d) providing an insulin administration device to administer insulin to the person with diabetes,
e) providing a device for automatically filling the insulin administration device,
f) communicating the calculated dose from the protocol manager to the filling device and using the filling device to fill the insulin administration device with the calculated dose,
g) administering the calculated insulin dose to the person with diabetes using the filled insulin administration device.

2. The method of claim 1 wherein the insulin administration device is capable of providing a relatively constant insulin concentration in the blood of the person with diabetes.

3. The method of claim 2 wherein the insulin administration device is a syringe.

4. The method of claim 3 wherein the insulin is long acting insulin.

5. The method of claim 1 wherein the insulin administration device is capable of delivering insulin to the body at a relatively constant rate.

6. The method of claim 5 wherein the insulin administration device is a body adhesive patch.

7. The method of claim 6 wherein the insulin is regular or fast acting insulin.

8. The method of claim 1 wherein the filling device is comprised of two components where the first component comprises a container of a relatively high fixed concentration of insulin and a container of diluent and the second component comprises a metering device capable of aliquoting the concentrated insulin and the diluent to provide a selected dose of insulin in a fixed volume of fluid.

9. The method of claim 8 wherein the first component is disposable and mates with a reusable second component to resupply the system with the necessary solutions to continue the method.

10. The method of claim 8 wherein the range of doses is 10 units to 150 units of insulin.

11. The method of claim 10 wherein the volume of the dose for filling the insulin delivery device is constant for all insulin doses.

12. The method of claim 1 wherein the insulin administration device is adapted to mate with the filling device in an aseptic manner.

13. The method of claim 8 wherein the first component mates with the second component in an aseptic manner.

14. The method of claim 1 wherein the measure of glycemic control is a fasting blood glucose measurement or fasting interstitial fluid glucose measurement.

15. The method of claim 1 wherein the measure of glycemic control is a fructosamine measurement.

16. The method of claim 1 wherein the measure of glycemic control is a hemoglobin A1C measurement.

17. The method of claim 1 wherein the measure of glycemic control is a postprandial blood glucose measurement or postprandial interstitial fluid glucose measurement.

18. The method of claim 1 wherein the communication of the measurement of glycemic control is made on a periodic basis.

19. The method of claim 18 wherein the periodic basis is daily or once every three days.

20. The method of claim 1 wherein the communication of the measurement of glycemic control is made using a telephone.

21. The method of claim 1 wherein the communication of the measurement of glycemic control is made using the internet or world wide web.

22. The method of claim 1 wherein the protocol manager is one or more persons.

23. The method of claim 1 wherein the protocol manager comprises computer software.

24. The method of claim 1 wherein the measure of glycemic control is provided using a keypad or wireless communication from a glucose meter.

25. The method of claim 1 wherein the measure of glycemic control is provided using voice recognition software.

26. The method of claim 1 wherein the filling device comprises the device for receiving and communicating the measurement of glycemic control.

27. The method of claim 8 comprising the further steps of a) using the calculated doses of insulin and the quantity of insulin contained in the dispensing device to determine when additional insulin is needed by the user and b) communicating a notice of resupply need to an appropriate resupply organization.

28. A method of treating a person with diabetes comprising the steps of
a) obtaining a measurement of glycemic control from the person with diabetes,
b) communicating the measurement of glycemic control to a protocol manager,
c) using the protocol manager to calculate a dose of insulin based on the obtained glycemic control measurement and previously communicated glycemic control measurements if earlier obtained,
d) communicating the calculated insulin dose to the person with diabetes, and
e) administering the calculated insulin dose to the person with diabetes.

29. A method of treating a person with diabetes comprising the steps of
a) obtaining a measurement of glycemic control from the person with diabetes,
b) communicating the measurement of glycemic control to a protocol manager,
c) using the protocol manager to calculate a dose of insulin based on the obtained glycemic control measurement or the obtained glycemic control measurement and previously obtained glycemic control measurements,
d) filling an insulin administration device with the calculated dose of insulin or adjusting a prefilled insulin administration device to deliver the calculated dose, and
e) administering the calculated insulin dose to the person with diabetes using the insulin administration device.
